(19) United States
(12) Patent Application Publication
(10) Pub. No.: US 2007/0021841 A1
(43) Pub. Date: Jan. 25, 2007

(54) Joint for prosthesis

(75) Inventors: Ihsan Al-Temen, Richmond Hill (CA); David L. Wells, Etobicoke (CA); Gillbert Chau, Scarborough (CA)

Correspondence Address: TORYS LLP, 79 WELLINGTON ST. WEST, SUITE 3000, TORONTO, ON, M5K 1N2, CA

(73) Assignee: Bloorview Kids Rehab, a corporation registered under the Ontario Corporations Act, Toronto (CA)

(21) Appl. No.: 11/384,333

(22) Filed: Mar. 21, 2006

Related U.S. Application Data
(60) Provisional application No. 60/664,906, filed on Mar. 24, 2005.

(51) Int. Cl. A61F 2/72; A61F 2/80; A61F 2/54

(52) U.S. Cl. 623/25; 623/57; 623/36

(57) ABSTRACT

A prosthesis is provided having a joint that allows for substantially quick-connection and quick-disconnection of a shell to a liner. The joint also allows for rotational movement and effects electrical connection and mechanical connection between the shell and the liner during connection of the shell to the liner and maintains the connection throughout rotation of the shell in relation to the liner.

### PRIORITY CLAIM

The present application claims priority from U.S. Provisional Patent Application 60/664,906, filed Mar. 24, 2005, the contents of which are incorporated herein by reference.

### FIELD OF THE INVENTION

The present invention relates generally to field prosthetics and more particularly relates to joints for prostheses.

### BACKGROUND OF THE INVENTION

Prostheses can be an important part of providing greater physical independence for individuals who have lost limbs. Silicone liners have proven to be an effective way to suspend upper and lower-extremity prostheses. However, this type of fitting has been problematic for the upper extremity myoelectric application. More specifically, where an individual has lost a forearm, a prosthesis liner can be fit over the individual's upper arm, and a shell containing the mechanical limb connected to the liner. One problem with prior art prostheses, at least of this type, is that the mechanical joints must provide a sufficient degree of rotation, while also serving as an electrical joint for a plurality of conductors. As a result, conductors need to be elegantly bundled in order to maintain their integrity during mechanical rotation of the prosthesis.

It is known in prosthetic wrists to provide a contact plate that can achieve mechanical rotation while preserving electrical contact. For example, The Contact Plate from Otto Bock HealthCare GmbH, Max-Näder-Str. 15, 37115 Duderstadt, Germany, and identified by part number 9E371 is an element of the Otto Bock Electrohand 2000. It can provide passive wrist rotation, and centralized, constant friction electrical contact. seen in Otto Bock Quality for Life Catalogue—MYOBOCK Arm Components, 2005, page 6.8 However, this Contact Plate is generally unsuitable for silicone sleeve prostheses because it does not provide the appropriate support to enable both mechanical suspension and electrical connection. Also, it does not provide for a fixed and stable rotational position about the axis of the prosthetic shell.

### SUMMARY OF THE INVENTION

It is an object of the present invention to provide a novel prosthesis that obviates or mitigates at least one of the above-identified disadvantages of the prior art.

A prosthesis with an improved suspension and electronic interconnect is provided. The prosthesis can enable individuals to use powered, upper-extremity prostheses and achieve increased range of motion.

An aspect of the invention provides mechanism providing electrical and mechanical connection at a prosthetic joint. The mechanism can be implemented so as to provide the connection in a quick-connect format whereby two parts can be “snapped” together to provide both mechanical and electrical connection simultaneously and as part of the same quick-connect unit. The mechanism can be implemented with the quick-connect unit such that the connection can be easily released by the push of a button to separate the two parts. The mechanism can be implemented whereby the quick-connect is integrated with a silicone sleeve prosthetic liner. Alternatively, or in addition, the mechanism can be modified with connect like a coaxial plug. The mechanism can be modified to provide any a number of connection traces/sites for electrical connections. The mechanism can be modified to provide processing electronics and/or circuit boards containing connection traces.

Aspects include a novel children's upper-limb prosthesis. In one aspect, the prosthesis can be fit onto a young child has been successfully fit using a silicone liner and remote electrodes. The prosthesis can overcome or mitigate a number of prior art limitations, including wire breakage, difficulty of electrode and wire attachment, and difficulty of donning/doffing of the prosthesis because the electrodes were hard-wired across the mechanical connection.

Embodiments can provide an electromechanical quick-connect attachment that enables the user to easily connect/disconnect the prosthetic shell and hand system to/from the liner, yet still provide mechanical suspension/connection between liner and prosthesis and provides for electrical connection of sensor electrodes with control electronics. Electrical, mechanical and functional issues are addressed in the development of the attachment. Issues included robustness of the connection, reduction and/or minimization of electrical noise, strength and durability of the connection, and ease of use and proper suspension of the prosthesis. The quick-connect prosthesis can be applicable for many control input methods. For adults and children with upper-extremity limb loss, this technology enables the benefits of a prosthesis with a better range of motion than traditional “hard shell” systems.

### BRIEF DESCRIPTION OF THE DRAWINGS

The invention will now be described by way of example only, and with reference to the accompanying drawings, in which:

FIG. 1 is an isometric view of a prosthesis in accordance with an embodiment of the invention;

FIG. 2 is a side sectional view of a portion of the prosthesis of FIG. 1;

FIG. 3 is a side sectional view of a portion of the prosthesis of FIG. 2 when assembled;

FIG. 4 is an isometric view of the joint of the prosthesis from FIG. 2 when the disassembled;

FIG. 5 is a side sectional view of the joint of the prosthesis from FIG. 2 when assembled;

FIG. 6 is a side sectional view of the joint of the prosthesis from FIG. 2 when disassembled;

FIG. 7 is an isometric view of a portion of the electrical connections from joint from FIG. 4;

FIG. 8 is an isometric view of a portion of the mechanical connections from joint from FIG. 4;

FIG. 9 is a front view of a slip ring from the female portion of the joint shown in FIG. 4;

FIG. 10 is a rear view of a slip ring from the female portion of the joint shown in FIG. 4.

### DETAILED DESCRIPTION OF THE INVENTION

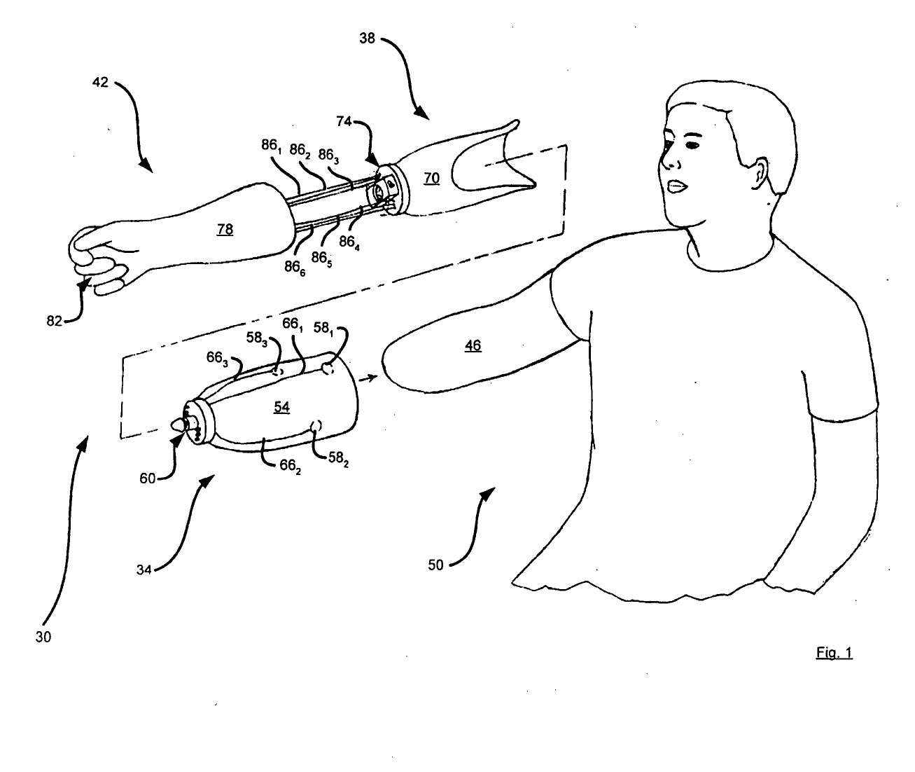

Referring now to FIG. 1, a prosthesis in accordance with an embodiment of the invention is indicated generally at 30. Prosthesis 30 comprises a liner 34 for complementary engagement with a shell 38. Prosthesis 30 also includes a sleeve 42 which covers shell 38 and liner 34 when prosthesis 30 is assembled and worn on an upper arm 46 of an individual 50.

As seen in FIGS. 1-3, liner 34 has a hollow body 54 for receiving upper arm 46 therein. Body 54 is thus made from any suitable material such as silicone and is shaped for a complementary fit over upper arm 46. Three electrodes 58₁, 58₂ and 58₃ are provided along body 54. (Collectively, electrodes 58₁, 58₂ and 58₃ are referred to as electrodes 58, and generically as electrode 58. This nomenclature is used for other components discussed herein.) Electrodes 58 are located along body 54 so that electrodes 58 contact certain tissues on upper arm 46. Such tissues are selected so that individual 50 can deliver biological impulses those tissues, which in turn can be registered as electrical impulses by electrodes 58, in order to control movement of prosthesis 30. Liner 34 also includes a male joint 62 which connects to each electrode 58 via respective conductor-pairs 66. Such conductor-pairs 66 can be shielded, if desired, to reduce electrical noise. Conductor-pairs 66 can also be made from a stretchable material so they can accommodate stretching or other stresses with reduced likelihood of breakage. Male joint 62 will be discussed in greater detail below.

Shell 38 includes a housing 70 for receiving liner 34 therein. Housing 70 is thus made from any suitable material such as glass-filled nylon and is shaped for a complementary fit over liner 34. Shell 38 also includes a female joint 74 which electrically and mechanically connect to male joint 62. Collectively, female joint 74 and male joint 62 provide a joint between liner 34 and shell 38. Female joint 74 will be discussed in greater detail below.

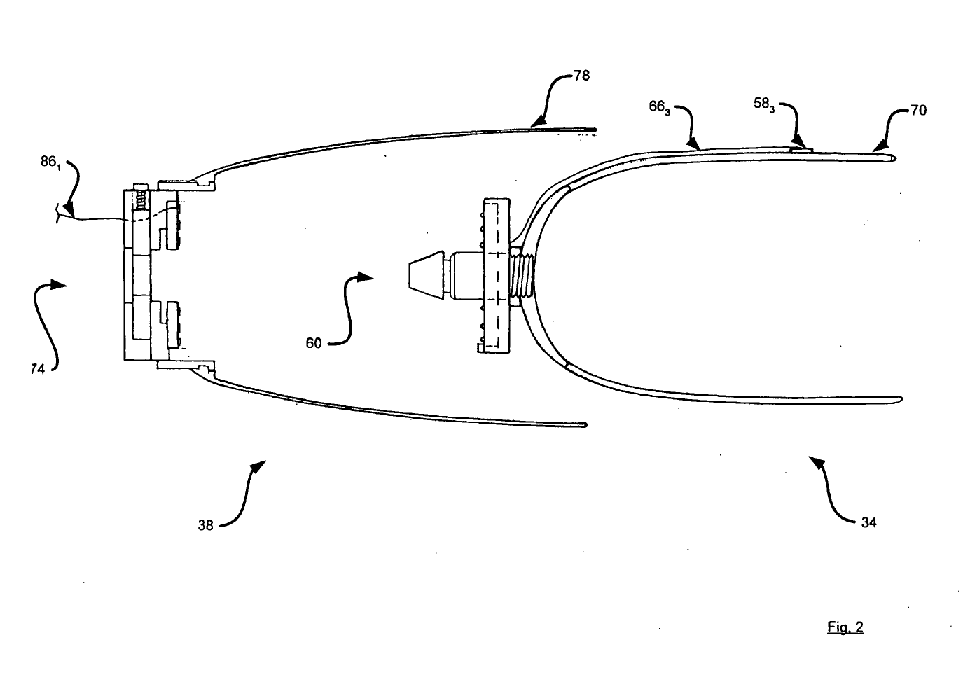

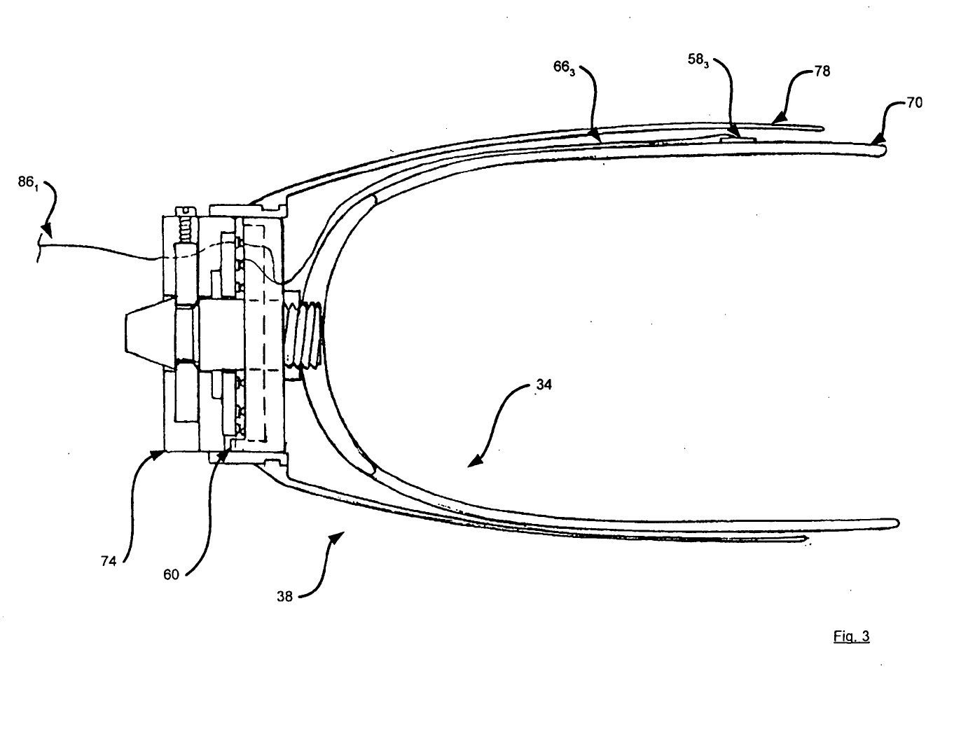

Referring back to FIG. 1, sleeve 42 includes a silicone (or other suitable material) outer layer 78 and includes an artificial hand 82 at the distal end of sleeve 42. Sleeve 42 can be pulled over shell 38 and liner 34 when shell 38 and liner 34 are fit over upper arm 46, thereby fully assembling prosthesis 30 to individual 50. Sleeve 42 contains electromechanical workings for effecting movements offered by prosthesis 30. The configuration and type of such workings are not particularly limited, and thus are not shown in detail and need not be discussed further herein. Such workings, however, are connected to female joint 74 via conductors 86, in order to receive electrical impulses from respective electrodes 58. (For convenience, only one conductor-pair 66 and only one conductor 86 is shown in FIGS. 2 and 3).

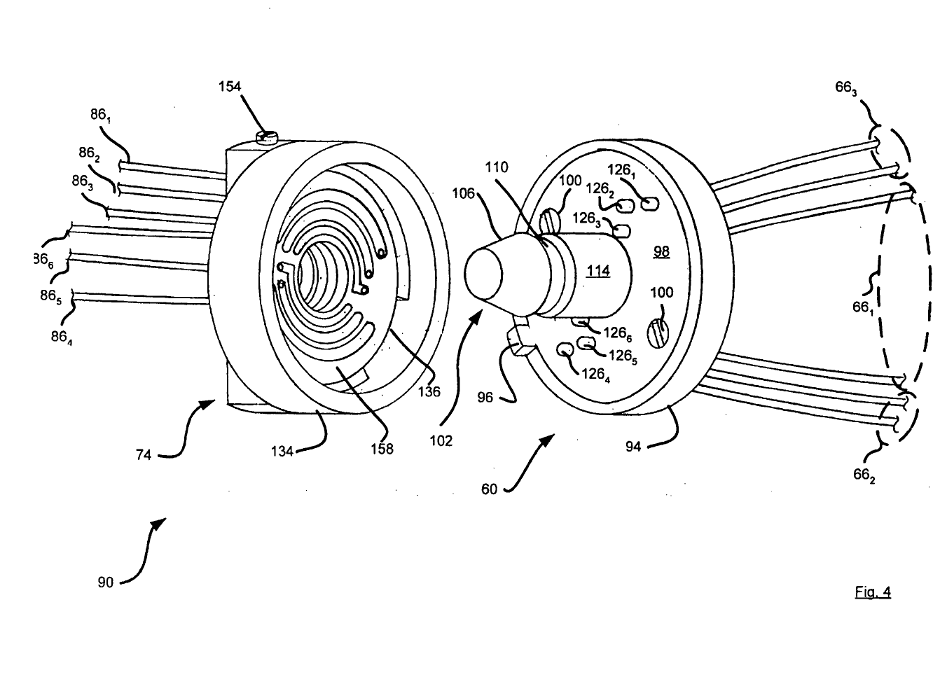

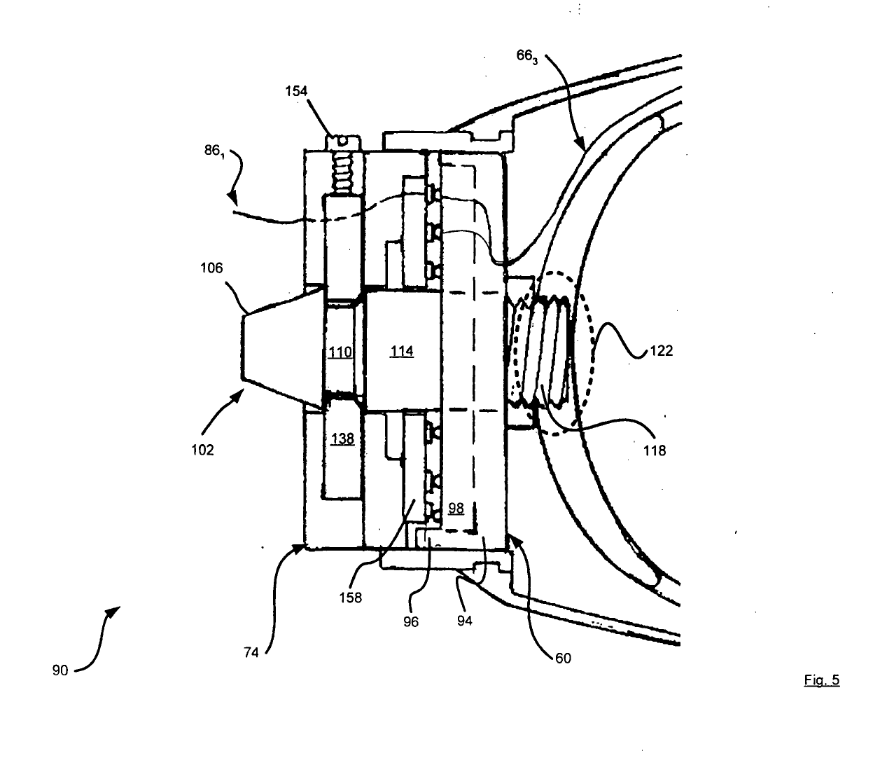

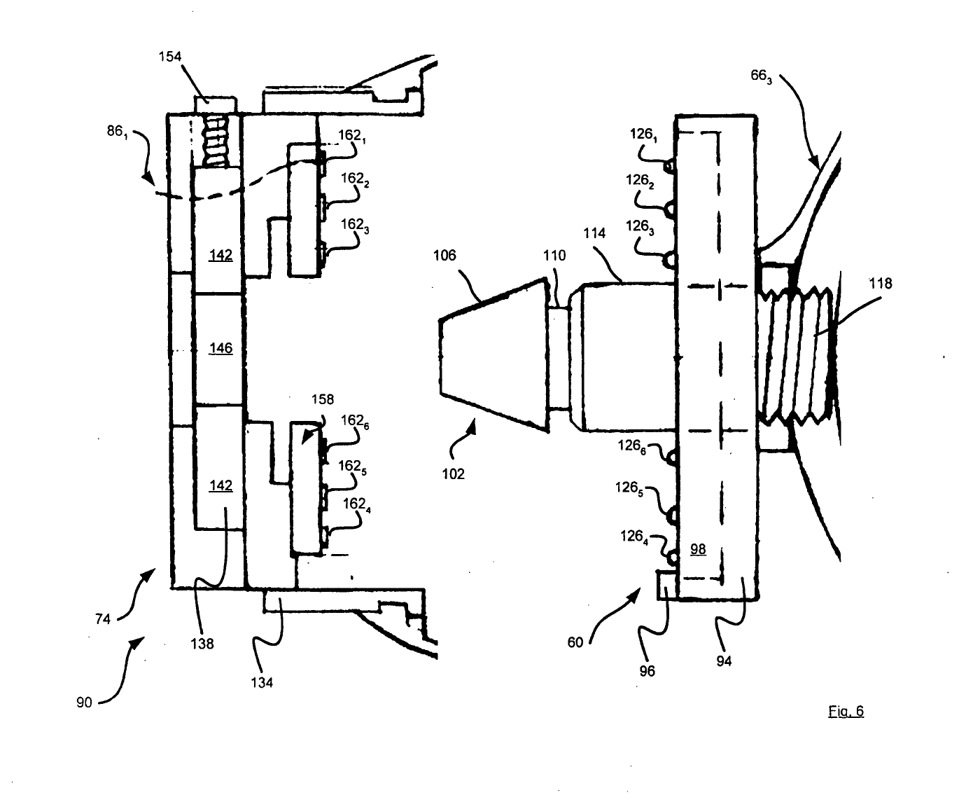

Referring now to FIGS. 4-6, male joint 60 and female joint 74 are shown in greater detail. Hereafter, and as indicated on FIGS. 4-6, male joint 60 and female joint 74 are collectively referred to as joint 90. (For convenience, only one conductor-pair 66 and only one conductor 86 is shown in FIGS. 5 and 6).

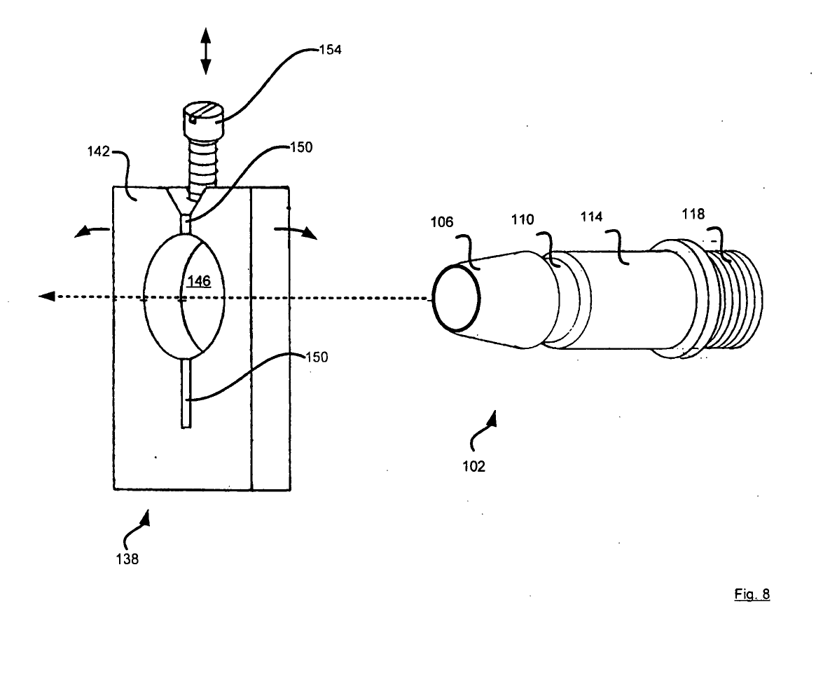

Male joint 60 comprises a hollow disc 94 that acts as a chassis for male joint 60 and supports a cover plate 98. Disc 94 is substantially uniform along its periphery, but in present embodiment includes a tab 96 that projects away from liner 34 towards female joint 64. Cover plate 98 is fastened to disc 94 by a pair of screws 100, but it is to be understood that in other embodiments other fastening means can be used. A shaft 102 protrudes from disc 94 away from conductor-pairs 66 towards female joint 74. (Shaft 102 is shown in detail in FIG. 8, however, the features of shaft 102 are also shown in FIGS. 4-6). Shaft 102 includes a truncated conical head 106 that is narrow at the distal tip. Head 106 widens along the length towards cover plate 98. Shaft 102 also includes a neck 110, which lies intermediate head 106 and a body 114 of shaft 102. Neck 110 provides a groove that is narrower than portions of head 106 and body 114 that are directly adjacent to neck 110. Body 114 is substantially cylindrical and extends through disc 94. A externally threaded portion 118 characterizes the portion of body 114 that is opposite neck 110. Threaded portion 118 is thus configured for secure fastening of male joint 70 to liner 34 via an internally threaded cavity 122 disposed within the central tip of housing 70 of liner 34.

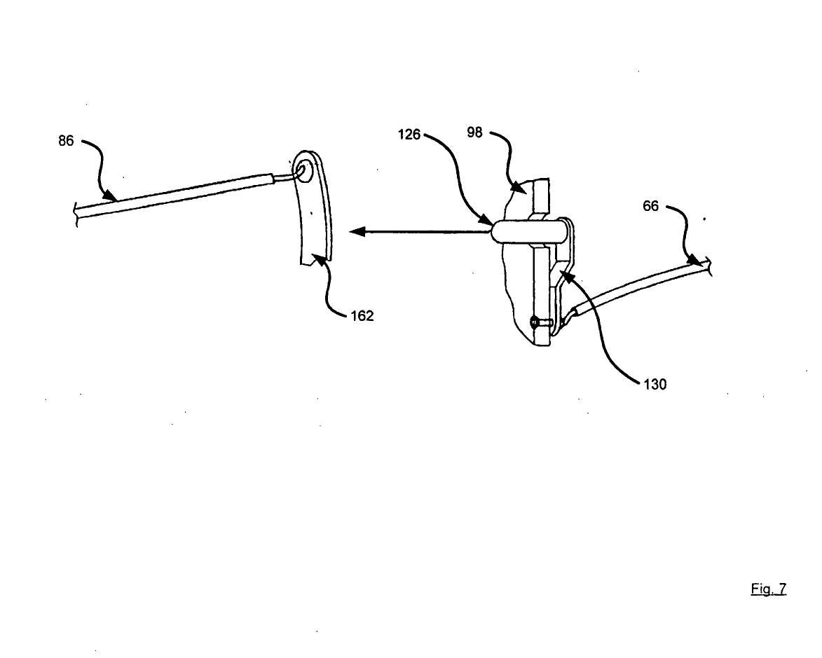

Male joint 60 further comprises a plurality of pins 126, pairs of which are connected to conductor-pairs 66. Specifically, in the present embodiment, pins 126₁, and 126₂ are connected to conductor-pair 66₃; pins 126₃ and 126₆ are connected to conductor-pair 66₁; and pins 126₄ and 126₅ are connected to conductor-pair 66₂. As can be best seen in FIG. 7, each pin 126 protrudes through a respective opening within cover plate 98. Again, as seen in FIG. 7, each pin 126 is affixed to cover plate 98 via a spring 130, which is biased towards cover plate 98 and thereby urging each pin away from cover plate 98 and towards female joint 74. Each spring 130 is also connected to one of the conductors in each conductor-pair 66, thereby providing the electrical connection from each electrode 58 to its respective pin 126.

Referring again to FIGS. 4-6, female joint 74 comprises a skirt 134 shaped to fit over disc 94. Skirt 134 includes a groove 136 for receiving tab 96 on disc 94. Tab 96 and groove 136 are so paired to act as a guide so that when female joint 74 is assembled to male joint 60, tab 96 will be inserted into groove 136 and thereby provide the proper electrical connections for joint 90. Tab 96 and groove 136 also cooperate to provide mechanical stops that restrict the range of rotation. The length of groove 136 is chosen to effect a range of rotation that is substantially the same range of rotation that is available in the human arm. The details of such electrical connections will be discussed further below.) Skirt 134 is a chassis that supports a collar 138.

As best seen in FIG. 8, collar 138 is comprised of a rectangular block 142 with an central opening 146 and a split 150 on either side of opening 146. Block 142 is thus biased such that opening 146, in a first position, has a first diameter that is smaller than the widest portion of head 106, but larger than neck 110. However, halves of block 142 can be urged outwardly such that opening 146 expands to receive head 106 therethrough. Due to the tapered shape of head 106, head 106 will urge the halves of block 142 outwardly to accommodate the widest portion of head 106. However, once head 106 has passed through opening 146, opening 146 returns to its first diameter and encircles neck 110. Collar 138 also includes a set-screw 154 which can be adjusted in order to set the degree to which shaft 102 is securely retained by collar 138.

In general terms, collar 138 is configured to receive shaft 102 and then secure shaft 102 to collar 138. (And it should be understood that other configurations of are contemplated to achieve this result.) Thusly, male joint 60 can be mechanically secured to female joint 74, and likewise securing shell 38 to liner 34 while allowing shell 38 to mechanically rotate about liner 34. By the same token, by adjusting set-screw 154 to the appropriate setting, removal of shell 38 from liner 34 can be effected by applying appropriate angular pressure to urge opening 146 into a larger size and allow shaft 102 to be removed from collar 138.

In general, the mechanical engagements between shell 38 and liner 34 are configured to have a level of friction therebetween to reduce rotational slippage between said liner and said shell. One significant place within the mechanical engagements where such friction can occur is between collar 138 and shaft 102. The level of friction can be chosen such that force of gravity acting alone on shell 38 is insufficient to effect said rotational movement in relation to liner 34.

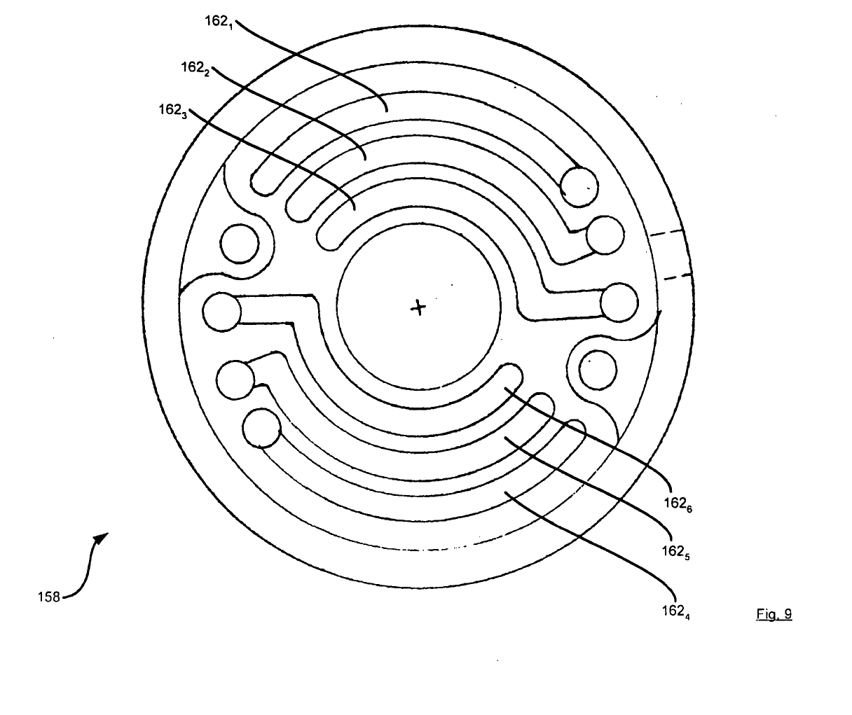

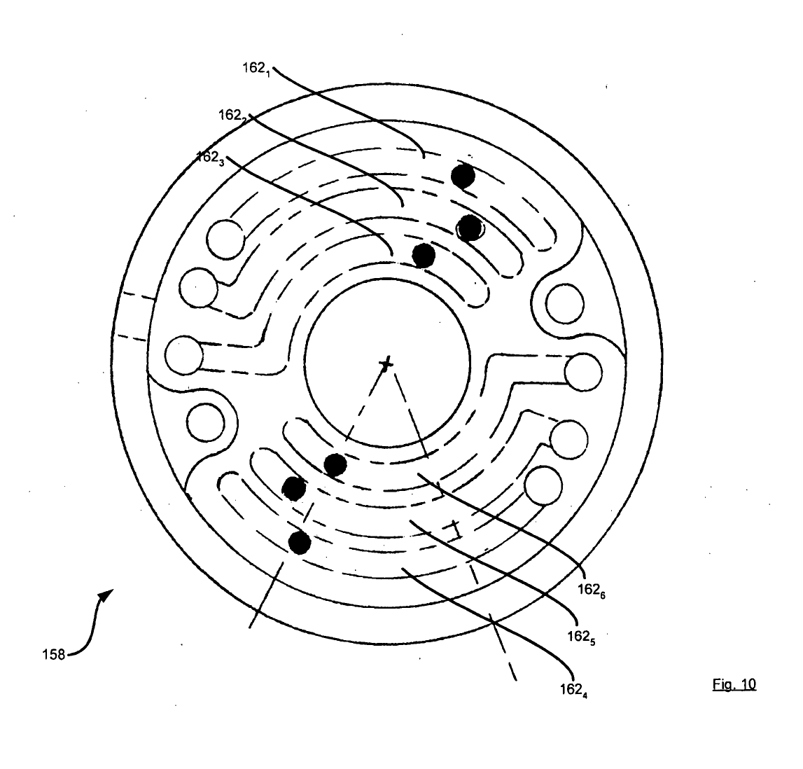

Skirt 134 also supports a slip ring 158 that is located the side of skirt 134 closest to male joint 60. (Slip ring 158 is shown in detail in FIGS. 9 and 10. FIG. 9 shows the side of slip ring 158 that faces male joint 60, while FIG. 10 shows the side of slip ring 158 that faces collar 138. Features of slip ring 158 are also shown in FIGS. 4-6). Slip ring 158 includes a plurality of traces 162 that are respective to each pin 126 and each conductor 86, such that engagement between a pin 126 and its respective trace 162 provides an electrical connection between a respective conductor 86 and a respective half of a conductor pair 66. (This is illustrated in FIG. 7). Traces 162 can be made of any suitable conducting material such as aluminum. Which ever material is chosen, a gold-coated can also be applied to traces 162 to reduce electrical noise. Other coatings can also be applied to reduce resistance and/or wear of traces 162.

In use, it is assumed that sleeve 42 is drawn over shell 38 and are thus assembled together, but that liner 34 is not assembled to the remaining components of prosthesis 30. Referring to FIG. 1, in use liner 34 is placed over upper arm 46 of individual 50. Next, as shown in FIG. 2, shell 38 is placed over liner 34 and female joint 76 is drawn towards male joint 60. As best seen in FIG. 8, as female joint 76 comes into contact male joint 60, opening 146 of collar 138 is placed over head 106 of shaft 102, and head 106 urges block 142 outwardly so that head 106 can pass completely through opening 146. Having done so, and as best seen in FIGS. 3 and 5, collar 138 will now encircle neck 110, thereby mechanically securing female joint 76 to male joint 60, and thereby securing shell 38 to liner 34.

By the same token, shell 38 can be removed from liner by applying a force along shell 38 in a direction normal to liner 34, thereby enlarging 146 as neck 110 pushes against block 142, and then applying a force on shell 38 that is away from Where individual 50 has full use of the arm opposite upper arm 46, it is contemplated that at least some individuals will be able to accomplish connection and disconnection of shell 38 to liner 34 using that opposite arm, and in a manner that is at least somewhat easier than prior art prostheses. The result of the foregoing is that a substantially quick-connect and substantially quick-disconnect of shell 38 to liner 34 can be effected.

Having affixed prosthesis 30 to individual 50, individual 50 can then deliver biological impulses to tissue in contact with electrodes 58. In turn, electrical signals are delivered from electrodes 58, along conductor-pairs 66 to respective pins 126, and then transmitted to their respective traces 162, and then in turn transmitted to conductors 86, to the electromechanical workings of sleeve 42. Such electrical signals are then used by those electromechanical workings to cause, in the usual manner, movements in hand 82 and prosthesis 30 in general. As such movements are effected, mechanical rotation of sleeve 42 and shell 38 in relation to liner 34 occurs about shaft 102; simultaneously, electrical communication between conductor-pairs 66 and respective pairs of conductors 86 is maintained as pins 126 are urged into abutment with respective traces 162, and throughout the travel along those trances 162 throughout the range of rotational motion offered by joint 90.

While only specific combinations of the various features and components of the present invention have been discussed herein, it will be apparent to those of skill in the art that desired subsets of the disclosed features and components and/or alternative combinations of these features and components can be utilized, as desired. For example, the electromyographic sensor described herein can be modified for use with a plurality of different types of man machine interfaces, including prosthetic limbs, computing pointing devices, etc.

As another example, while substantially quick-connect and substantially quick-disconnect of shell 38 to liner 34 is effected using the particular joint 90 described above, other joints 90 can be configured as well that accomplish substantially quick-connect and substantially quick-disconnect. For example, shaft 102 can be associated with shell 38 and collar 138 associated with liner 34. By the same token, pins 126 and traces 162 can be reversed. Combinations thereof are also possible.

As an additional example, six conductors are used in prosthesis 30. The use of six conductors, and a presently preferred desired range of rotational movement results in the particular configuration of traces 162 shown in FIGS. 9 and 10 and previously mentioned mechanical stops. It should be understood that any pattern of traces, and number of traces, can be patterned onto slip ring 158 according to the desired number of conductors for prosthesis 30, subject to any physical limitations such as gauge of conductor, thickness of traces, electromagnetic interference effect and the like. In general, those of skill in the art will appreciate that the range of rotational motion offered by prosthesis 30 is about one-hundred-and-twenty degrees, but that other ranges of motion can be provided as desired according to the chosen configuration of joint 90 in general and, for example, slip ring 158 in particular. An example of another range of rotation is about ninety degrees.

As another example, prosthesis 30 is configured as a facsimile of a forearm, but could also be configured for other limb facsimiles, such as a lower leg.

In other variations, electrical components such as amplifiers and circuit boards can be incorporated into liner 34 or shell 38. Such components can be for noise suppression, pre-amplification, thresholding, etc.

In still further variations the mechanical stops and the friction within joint 90 can be chosen to accommodate desired needs and/or desired trace patterns.

The present invention provides a novel prosthesis. The prosthesis can make m donning/doffing more reliable and easier. The arc subtended by the connection trace can allow for contact within an allowed angular range of attachment, making it easier for the user to successfully don the prosthesis. Once the prosthesis is attached the connection elements do not rotate relative to each other, enabling better positioning control of the prosthesis. (i.e., once connected the parts don't slip relative to one another, creating positional uncertainty)

Increased reliability of electrical connection by reducing and/or avoiding and/or eliminating traditional across-the-joint hardwiring. In the prosthesis, mechanical and electrical connections are integrated together significantly reducing size and bulk (previously, systems were hardwired, and the mechanical connection was separate). The system can provide a foundation for the inclusion of additional processing electronics which can improve performance

The above-described embodiments of the invention are intended to be examples of the present invention and alterations and modifications may be effected thereto, by those of skill in the art, without departing from the scope of the invention which is defined solely by the claims appended hereto.

1. A prosthesis comprising:
a liner having a hollow body for receiving an upper limb therein and a first joint portion on a distal end of said body; said liner having a plurality of electrodes along said body for contacting tissue on said limb; said liner having a plurality of liner-conductors for carrying electrical signals from said tissue to said first joint portion;
a shell complementary to said liner and including a housing for receiving said liner therein and second joint portion on a distal end of sad shell; said shell having a plurality of shell-conductors for carrying electrical signals to electromechanical workings associated with said shell; and,
said joint portions having complementary mechanical engagements for substantially quick-connect and substantially quick-disconnect of said joint portions, such that when said joint portions are connected said mechanical engagements provide a rotational movement between said liner and said shell.

2. The prosthesis of claim 1 where said upper limb is an upper arm.

3. The prosthesis of claim 1 where said first joint portion is a male joint portion and said second joint portion is a female joint portion.

4. The prosthesis of claim 1 where said mechanical engagement of one of said joint portions includes a shaft and said mechanical engagement of a second of said joint portions includes a collar for releasably grasping said shaft while allowing rotation of said collar in relation to said shaft when said collar grasps said shaft.

5. The prosthesis of claim 4 where said collar includes an opening that is biased towards a first diameter and said shaft includes a head portion larger than said first diameter and for urging said opening towards a larger diameter such that said head can pass through said opening; said shaft including a neck smaller than said first diameter such that when said collar surrounds said neck said shaft is rotatable within said collar while said shaft is retained within said collar.

6. The prosthesis of claim 5 where an application of a force to said shaft normal to said collar urges said opening towards said larger diameter such that said shaft can be removed from said collar.

7. The prosthesis of claim 1 where said mechanical engagements are configured to have a level of friction therebetween to reduce rotational slippage between said liner and said shell.

8. The prosthesis of claim 7 where said level of friction is chosen such that force of gravity acting alone on said shell is insufficient to effect said rotational movement.

9. The prosthesis of claim 1 further comprising mechanical stops within said joint portions such that a range of said rotational movement is about one-hundred-and-twenty-degrees.

10. The prosthesis of claim 1 further comprising mechanical stops within said joint portions such that a range of said rotational movement is about ninety-degrees.

11. The prosthesis of claim 1 further comprising said joint portions having complementary electrical engagements that effect an electrical communication between pairings of said liner-conductors and shell-conductors during said substantially quick-connect; said electrical engagements maintaining said electrical communication during said rotational movement.

12. The prosthesis of claim 11 where said electrical engagement of one of said joint portions includes a pin and said electrical engagement of a second one of said joint portions includes a slip ring having a trace respective to each said pin; said pin and said trace for carrying said electrical communication.

13. The prosthesis of claim 12 where a spring biases said pin towards said trace.

14. The prosthesis of claim 11 where said traces made of aluminum.

15. The prosthesis of claim 11 where said traces are gold-coated

16. The prosthesis of claim 11 where said traces are coated with a material that reduces resistance between said pins and said traces.

17. The prosthesis of claim 11 further comprising three of said electrodes and a pair of liner-electrodes for each said electrode.

18. The prosthesis of claim 11 further comprising at least one additional electrical component mounted to at least one of said shell and said liner.

19. The prosthesis of claim 11 where said conductor-pairs are shielded.

20. The prosthesis of claim 11 where said conductor-pairs are made from a stretchable material.

21. A prosthesis comprising:
a liner having a hollow body for receiving an upper limb therein and a first joint portion on a distal end of said body; said liner having a plurality of electrodes along said body for contacting tissue on said limb; said liner having a plurality of liner-conductors for carrying electrical signals from said tissue to said first joint portion;
a shell complementary to said liner and including a housing for receiving said liner therein and second joint portion on a distal end of sad shell; said shell having a plurality of shell-conductors for carrying electrical signals to electromechanical workings associated with said shell;
said joint portions having complementary mechanical engagements for substantially quick-connect and substantially quick-disconnect of said joint portions, such that when said joint portions are connected said mechanical engagements provide a rotational movement between said liner and said shell; and,
said joint portions having complementary electrical engagements that effect an electrical communication between pairings of said liner-conductors and shell-conductors during said substantially quick-connect; said electrical engagements maintaining said electrical communication during said rotational movement.
